(19) United States
(12) Patent Application Publication
(10) Pub. No.: US 2025/0364133 A1
(43) Pub. Date: Nov. 27, 2025

(54) METHOD AND SYSTEM FOR ENHANCED DETECTION AND COUNTING OF RESPIRATORY EVENTS USING MACHINE LEARNING MODELS

(71) Applicant: PRANAQ PTE. LTD., Taipei city (TW)

(72) Inventor: Ke-Wei Chen, Taipei city (TW)

(21) Appl. No.: 18/670,154

(22) Filed: May 21, 2024

(51) Int. Cl. G16H 50/20; G16H 50/30

(52) U.S. Cl. CPC G16H 50/20; G16H 50/30

(57) ABSTRACT

The present invention relates to a system and method for the enhanced detection and quantification of respiratory events, such as apneas and hypopneas, particularly useful in diagnosing and managing sleep-disordered breathing conditions. This system integrates sophisticated machine learning algorithms, specifically one-dimensional convolutional neural networks (1D CNNs), with advanced signal processing techniques to analyze physiological signals. It focuses on the detecting respiratory event with a localized portion of the input segment, a novel approach that increases specificity in event detection. The system segments physiological signals into overlapping segments, each analyzed by the machine learning model to generate a prediction score. A unique aspect of this invention is the application of a dual-threshold mechanism: a ‘Model threshold’ for initial event identification and a ‘Vote threshold’ for confirming events through an aggregate voting process of overlapping segment predictions. This innovative approach ensures high accuracy and reliability in event detection and counting.

### BACKGROUND OF THE INVENTION

### 1. Field of the Invention

The present invention pertains to the field of medical monitoring and diagnostics, specifically to an automated method and system for detecting and quantifying respiratory events using machine learning models, applicable to sleep studies and managing sleep-disordered breathing conditions.

### 2. Description of the Prior Art

Diagnosis of sleep apnea, a disorder characterized by breathing interruptions during sleep, is traditionally conducted through polysomnography (PSG). This involves monitoring and analyzing multiple physiological parameters to identify apneic events. Current methods for respiratory event detection rely heavily on manual annotation by trained professionals or threshold-based automated systems. These practices, however, present significant drawbacks. Manual scoring is time-consuming, costly, and introduces variability due to subjective interpretation. Automated methods, on the other hand, may not consistently capture the complexity of physiological signals, leading to inaccuracies in the detection of apnea and hypopnea events.

Furthermore, existing automated systems often struggle with the variability in signal quality and the presence of artifacts, which can skew the results. The limited capacity of these systems to adapt to individual patient variability can result in both false positives and false negatives. This is a significant concern in clinical diagnostics where the accurate quantification of respiratory events is critical for determining the severity of sleep apnea and guiding treatment decisions.

The use of advanced machine learning techniques offers potential improvements in the analysis of time sequence data from physiological signals. However, the application of these models in sleep study analysis requires careful consideration of event labeling, signal processing, and the handling of ambiguous data points (e.g., cases where the probability is 0.5). Despite advancements in this area, there remains a gap between the potential of machine learning algorithms and their practical implementation in clinical and home-based monitoring systems.

Therefore, there exists a clear need for a system that can more accurately and reliably detect and count respiratory events, reducing the dependency on manual scoring and improving upon the limitations of current automated detection algorithms. Such a system would ideally be capable of processing complex physiological signals with high precision, adapting to individual patient characteristics, and providing consistent results across different operating conditions. The desired system should also be able to address the ambiguities in event onset and termination, which are often sources of inconsistency in event detection and scoring.

### SUMMARY OF THE INVENTION

The present invention provides a novel method and system for the detection and quantification of respiratory events, designed to overcome the limitations inherent in current sleep apnea diagnostic techniques. At its core, the invention integrates advanced machine learning models with a strategic approach to signal analysis and event prediction, aiming to enhance the accuracy and reliability of respiratory event counting in both clinical and home monitoring settings.

In contrast to traditional methods that rely heavily on manual scoring or basic threshold-based algorithms, this invention employs a combination of localized event labeling and an ensemble of predictions from machine learning models to refine the detection process. This approach reduces the subjectivity and inconsistency often encountered in manual annotation.

Further, the invention includes segmenting physiological signal data into overlapping segments of a predetermined length. These segments are analyzed by the machine learning models to produce prediction scores indicating the probability of respiratory event occurrences. By concentrating on this specific window, the method enhances the specificity of event detection, avoiding the common pitfalls of mislabeling due to irrelevant data outside the critical event period.

Significant to the invention is the implementation of a voting mechanism that aggregates the prediction scores from multiple model analyses of overlapping segments. This voting process serves as a form of validation and confirmation, ensuring that only segments that consistently indicate the presence of an event across multiple predictions contribute to the final event count. The system establishes a ‘Model threshold’ to identify segments with a high probability of containing an event and a ‘Vote threshold’ that segments must surpass to be confirmed as true events.

The result is a robust system capable of providing a more accurate count of respiratory events. By leveraging the precision of machine learning and the power of aggregate analysis, the invention substantially improves the detection of apneas and hypopneas, facilitating more accurate diagnoses and more effective treatment planning. The system's design is inherently adaptable, capable of accommodating a range of machine learning models suitable for time sequence signal processing, which allows for flexibility and potential future improvements in model performance.

Overall, the invention represents a significant advancement in the field of sleep disorder diagnostics. It offers a sophisticated yet practical solution to the challenges of accurately identifying and counting respiratory events, thereby filling a critical need in the management of sleep apnea and related conditions. This invention not only promises to enhance the efficiency and effectiveness of sleep studies but also to provide a foundation for the development of next-generation sleep apnea diagnostic tools.

### BRIEF DESCRIPTION OF THE DRAWINGS

The objects, spirits, and advantages of the preferred embodiments of the present invention will be readily understood by the accompanying drawings and detailed descriptions, wherein:

FIG. 1 shows an overview of a system for detecting respiratory events in one embodiment of the invention.

FIG. 2 shows a flowchart of a method for detecting respiratory events in one embodiment of the invention.

FIG. 3 is a schematic illustrating the voting procedure for event detection in the proposed system.

FIG. 4 is an illustrative representation of the partial labeling strategy employed in the system for enhanced detection of respiratory events.

FIG. 5 presents a schematic diagram of a 1D Convolutional Neural Network architecture, which is an embodiment of the machine learning model utilized in the system.

FIG. 6 shows an overview of a system for detecting respiratory events in another embodiment of the invention.

FIG. 7 shows an overview of a system for detecting respiratory events in a third embodiment of the present invention.

FIG. 8 shows an overview of a system for detecting respiratory events in a fourth embodiment of the present invention.

### DETAILED DESCRIPTION OF THE INVENTION

This following description provides an exhaustive account of an innovative method and system for the detection and quantification of respiratory events, particularly tailored for the diagnosis and management of sleep-disordered breathing conditions such as sleep apnea. The invention is articulated around a synergistic integration of advanced machine learning algorithms and refined signal processing techniques, aiming to overcome the challenges and limitations prevalent in current diagnostic practices.

The core objective of this invention is to enhance the accuracy and reliability of respiratory event detection in a clinical or home setting, thereby facilitating more precise diagnoses and effective treatment plans for sleep apnea. Traditional methods, predominantly manual scoring and basic automated systems, have been found lacking in terms of accuracy, consistency, and efficiency. In response, the present invention employs a system that combines the precision of machine learning models, particularly one-dimensional convolutional neural networks (1D CNNs), to analyzing physiological signal data.

This system's novel approach is centered on two key innovations: the use of the partial labeling strategy, and the implementation of a voting mechanism that aggregates predictions from multiple models. By using part of input window for event labeling, it provides a more fine-grained event detection. Furthermore, the voting mechanism, which synthesizes the output from overlapping signal segments, enhances the reliability of the event count, ensuring that only the most probable events are considered in the final analysis.

The detailed description that follows elucidates each aspect of the invention, from the hardware components and physiological signals involved to the intricate workings of the machine learning models and the voting process. The aim is to provide a clear and comprehensive guide that enables those skilled in the art to replicate and utilize the invention, highlighting its practical applications and the advantages it offers over existing technologies in the field of sleep study analysis and respiratory event detection.

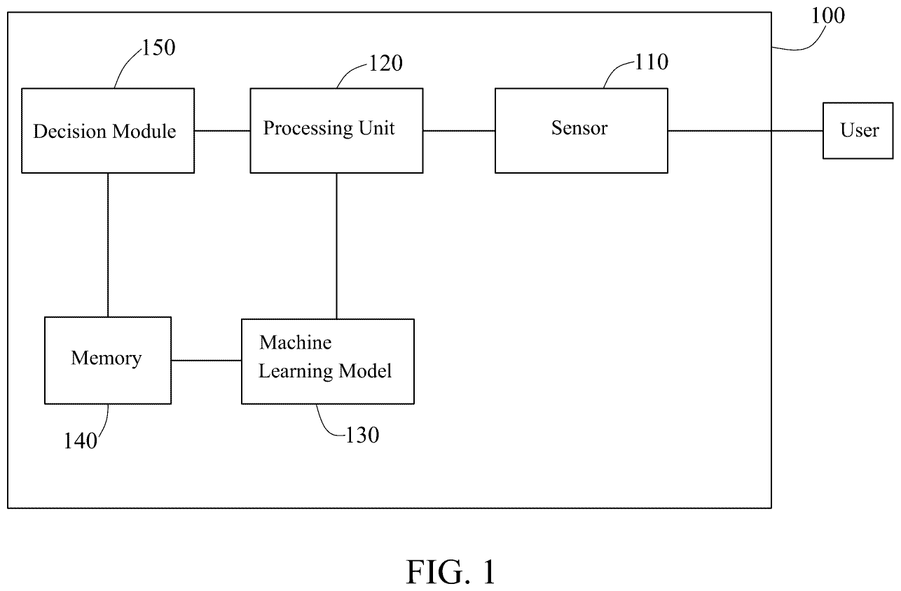

Please refer to FIG. 1. FIG. 1 shows an overview of a system in one embodiment of the invention. The system 100 described in this embodiment comprises several key components, each playing a significant role in the enhanced detection and counting of respiratory events. Central to the system 100 are the sensor 110, the processing unit 120, a machine learning model 130, a memory 140, and a decision module 150, all of which work in concert to capture, analyze, and interpret physiological signals.

At the forefront of the system are the sensor 110, which is such as a photoplethysmogram (PPG) sensor. The PPG sensor is adept at detecting blood volume changes in the microvascular bed of tissue, which can be reflective of respiratory activity due to the intrinsic relationship between respiratory and cardiovascular dynamics. The machine learning model 130 is specially configured to process the PPG signal. This configuration includes the extraction of features from the PPG signals that are correlated with respiratory events. The machine learning model 130 is trained to identify the subtle yet distinctive patterns that emerge in the PPG waveform due to the physiological impacts of breathing, such as variations in blood volume and flow associated with the respiratory cycle. These patterns may include but are not limited to changes in the amplitude of the PPG waveform, fluctuations in the timing of the peaks related to the heart rate, and the specific shape of the PPG waveform during different phases of respiration.

The processing unit 120 forms the backbone of the system 100, where the real-time data collected by the sensors are received and analyzed. This processing unit 120 is equipped with advanced computational capabilities to handle the complex algorithms and models (to be detailed below) required for processing the physiological signals. It is designed to be robust and efficient, capable of processing large volumes of data with high precision and speed.

The types of physiological signals processed by this system 100 are pivotal in detecting respiratory events. These physiological signals include peripheral oxygen saturation (SpO₂), instantaneous heart rate (IHR), and photoplethysmogram (PPG) envelope signals. SpO₂ is a measure of the oxygen level in the blood, a critical parameter that can be affected during apneic events. IHR provides insights into the heart's response to respiratory activities, while PPG envelope signals, derived from a photoplethysmogram, offer data on blood volume changes. The system 100 meticulously analyzes these physiological signals using its machine learning model 130 to detect subtle patterns and changes indicative of respiratory events such as apneas and hypopneas.

In this system 100, the memory 140 serves as the repository for significant data and parameters that are essential for the functioning of the machine learning model 130 and the decision module 150. Primarily, the memory 140 stores the ‘model threshold’ and the ‘vote threshold’ (to be detailed below).

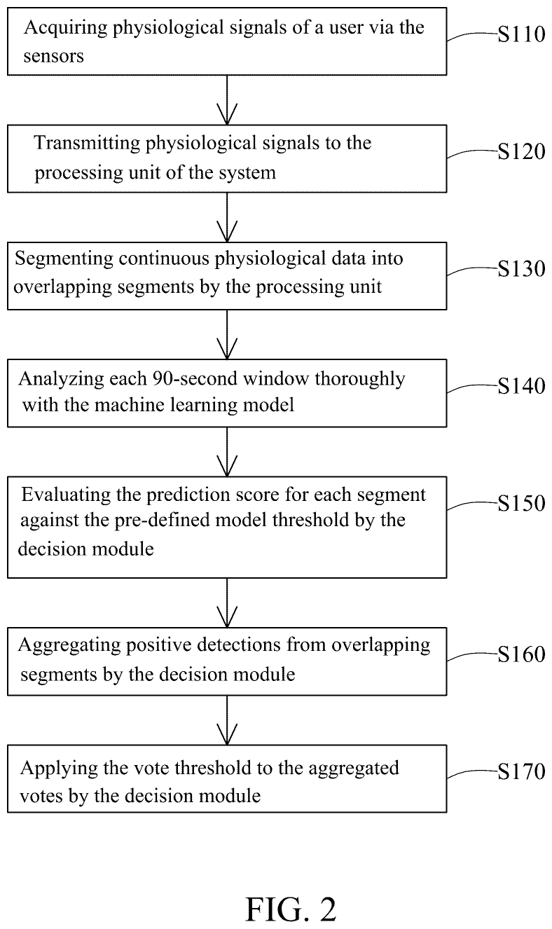

Please refer to both FIG. 1 and FIG. 2 simultaneously. FIG. 2 shows a flowchart of a method in one embodiment of the invention. Please refer to step S110. The process begins with the acquisition of physiological signals via sensor 110. These sensors 110 capture a comprehensive set of data, including peripheral oxygen saturation (SpO₂), instantaneous heart rate (IHR), and photoplethysmogram (PPG) envelope signals, which are critical for identifying respiratory events. Once these signals are captured, please refer to step S120; they are transmitted to the processing unit 120 of the system 110.

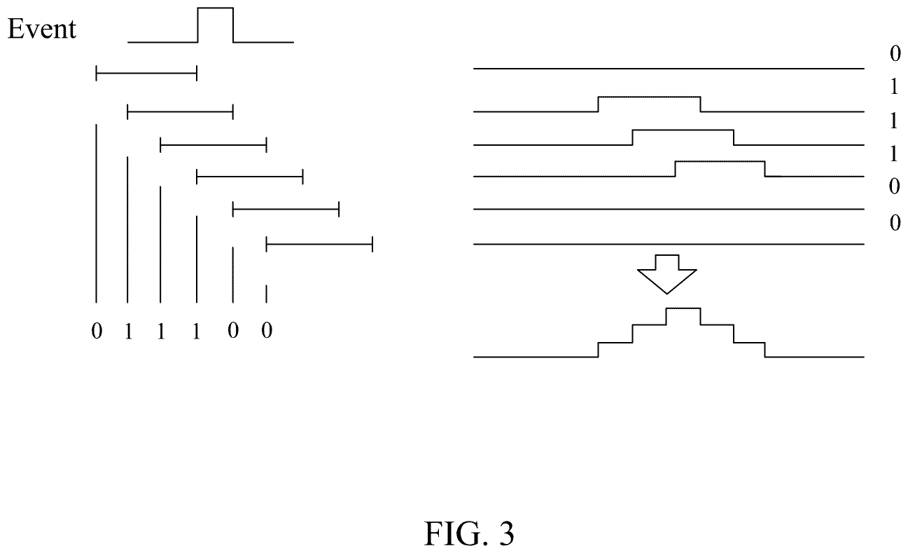

Upon receiving the signals, please refer to step S130, the processing unit 120 embarks on a significant stage—segmentation. Here, the continuous stream of physiological data is divided into overlapping segments. Each segment is defined to have a predetermined duration, typically set to typically set to range from 10 seconds to 30 minutes. This duration is chosen to balance the need for capturing enough data to accurately identify. For the next segment processing, the starting point of the next segment will move forward for a fixed step. This step size smaller than the predetermined duration (segment length) will be used, typically from 1 to 30 seconds. an event while maintaining manageable processing requirements. A notable aspect of this segmentation process is the overlap between consecutive segments (please refer to the left side FIG. 3). This extensive overlap ensures that no potential respiratory event is missed or partially captured at the boundaries of the segments.

Once segmented, please refer to step S140, each segments undergoes a thorough analysis by the machine learning model 130. The machine learning model 130, such as a 1D convolutional neural network (1D CNN), is adept at processing time-series data. The detection of an event will depend only on part of the input window, said the 20 seconds in the center of the segment. The machine learning model 130 evaluates the full input window to generate a prediction score for this center 20 second segment. This prediction score represents the probability of a respiratory event's presence in this partial segment. After generating a prediction score for each segment in step S140, the system proceeds to step S150. Here, the decision module 150 evaluates whether the prediction score for each segment exceeds the pre-defined model threshold. Segments that surpass this model threshold are marked as positive detections (indicated as “1” in FIG. 3), suggesting a high probability of a respiratory event.

Moving to step S160, please refer to the right side of FIG. 3 simultaneously. The decision module 150 then aggregates these positive detections from overlapping segments. This aggregation is a significant part of the voting mechanism, where each positive detection contributes a ‘vote’ towards the likelihood of a respiratory event in the specific time frame where these segments overlap. In the lower right corner of FIG. 3, the time frame with the highest number of votes represents the occurrence of the event.

Finally, in step S170, the decision module 150 applies the vote threshold to the aggregated votes. If the total number of votes within a specific time frame exceeds this threshold, the system confirms the occurrence of a respiratory event, which is then included in the final event count. This implementation of the voting mechanism by the decision module 150 ensures that respiratory events are accurately recognized, taking into account consistent indications across multiple segments, thereby enhancing the system's accuracy and reliability.

The decision module 150 in this system 100 can employ various algorithms or models to implement the voting mechanism and threshold application process. An example could be a rule-based algorithm where logical conditions are used to determine if the aggregated prediction scores meet the set ‘Vote threshold’. This could involve simple comparison operations (greater than or equal to the threshold) for each time frame across the overlapping segments.

Alternatively, the decision module 150 might use statistical models, such as Bayesian classifiers or decision trees, which consider the distribution of prediction scores and make decisions based on probabilistic reasoning or defined decision criteria. These statistical models are particularly effective in cases where decision-making involves assessing the likelihood of an event based on multiple data points and where the relationships between these data points are complex.

In brief, the decision module 150 can utilize a range of algorithmic approaches, from straightforward rule-based systems to more sophisticated statistical models, to effectively aggregate predictions and apply thresholds for accurate event detection.

In some embodiment, particular attention is paid to the adaptability of the vote threshold, which is a critical factor in the accurate detection of respiratory events. The decision module 150 is not only responsible for aggregating votes and applying the vote threshold but also for dynamically adjusting it. This adjustment is based on selected specificity and sensitivity criteria, which are essential in medical diagnostic applications. Specificity refers to the system 100's ability to correctly identify non-events (true negatives), while sensitivity pertains to correctly detecting true respiratory events (true positives). By calibrating the vote threshold in accordance with these criteria, the system 100 ensures a balanced and precise detection process. This calibration is facilitated through the analysis of extensive datasets and clinical trial results, allowing the system 100 to maintain high accuracy even under varying patient conditions and signal qualities. Such adaptability is key in tailoring the system's performance to specific clinical requirements and patient populations, thereby enhancing its overall efficacy in respiratory event detection.

The machine learning model 130's prediction score is not merely a binary output but a nuanced probability value that reflects the likelihood of an event's occurrence. For a segment to be considered positive, indicating the presence of a respiratory event, its prediction score must surpass a predefined threshold, known as the ‘Model threshold.’ This Model threshold is established based on historical data and expert input to optimize the balance between sensitivity and specificity.

The machine learning model 130 employed in this system 100, which in this embodiment is the one-dimensional convolutional neural network (1D CNN), are pivotal to the accurate and efficient detection of respiratory events. The choice of 1D CNN is grounded in their proven efficacy in handling time-series data, which is essential in the context of processing physiological signals for respiratory event detection.

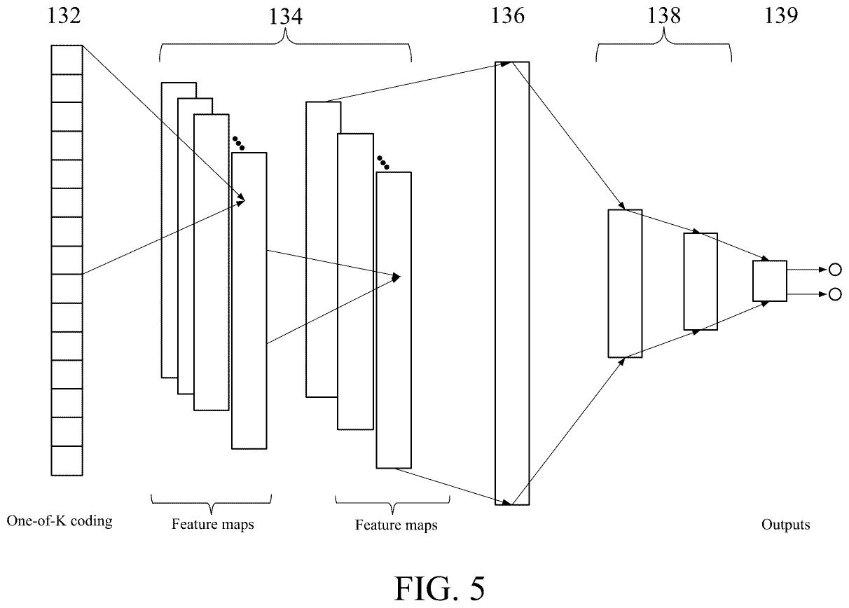

Please refer to FIG. 5, which delineates the 1D CNN architecture, constituting an embodiment of the machine learning model 130 within the system 100. This architecture is specifically tailored to analyze the time-series physiological data acquired by the sensors 110. The input layer 132 receives the one-of-K encoded signals, which are then processed through multiple convolutional layers 134, each designed to extract a diverse set of features through convolution operations. These features, represented as feature maps, are critical for identifying the nuanced patterns indicative of respiratory events. A flatten layer 136 subsequently condenses these feature maps into a format suitable for the dense layers 138 that follow, which further refine the features and contribute to the decision-making process. The culmination of this process is presented in the output layer 139, which generates a probabilistic score indicating the likelihood of a respiratory event's occurrence. This score forms the basis for subsequent decision-making steps in the system 100, as previously outlined in steps S140 to S170, demonstrating the integrated functionality of the 1D CNN within the broader context of the system's operation for respiratory event detection and counting.

The training process of the machine learning model 130 is a critical aspect of their functionality. Initially, the machine learning model 130 is trained on a large dataset comprising various examples of respiratory events, including both normal breathing patterns and events indicative of sleep apnea. This dataset is derived from diverse demographic groups to ensure robustness and generalizability of the machine learning model 130. During training, the machine learning model 130 learn to recognize patterns associated with different types of respiratory events, honing their ability to discern subtle anomalies indicative of apneic episodes.

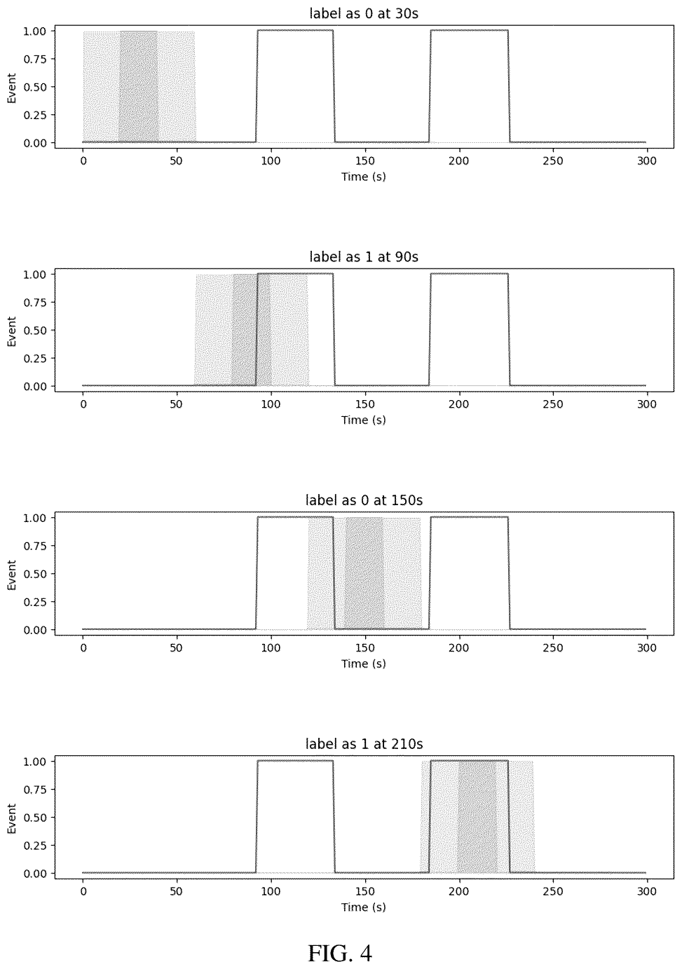

In applying the machine learning model 130 to the targeted window within each segment, a focused approach is adopted. As the example depicted in FIG. 4 which use 60 seconds as input segment length (as shown in ligth shading in FIG. 4), the machine learning model 130 concentrates on a 20-second window (as shown in darkest shading in FIG. 4) within this segment. A positive segment will only be defined when this 20 second window overlap the ground truth labeling (as shown with a solid line).

The machine learning model 130 analyzes this targeted window, applying its learned filters and kernels to extract relevant features. It then generates a prediction score for the segment, reflecting the likelihood of a respiratory event's occurrence within this window. By focusing on this specific part of the segment, the model effectively reduces the noise and potential for error that might arise from less relevant parts of the signal.

In essence, the machine learning model 130, particularly the 1D CNN, is carefully designed and trained to excel in the detection of respiratory events. Their architectural features, training methodologies, and targeted application to specific windows within the signal segments collectively ensure that the system 100 achieves high accuracy and reliability in respiratory event detection, a cornerstone of this innovative system.

In addition to the one-dimensional convolutional neural network (1D CNN), the system is adaptable to incorporate other sophisticated machine learning models based on the specific requirements of respiratory event detection and the characteristics of the physiological data being analyzed. These machine learning models include two-dimensional convolutional neural networks (2D CNNs), recurrent neural networks (RNNs), models with self-attention mechanisms, and decision-tree based models, each offering unique advantages and capabilities.

Two-dimensional Convolutional Neural Networks (2D CNNs) are particularly useful when the physiological data can be represented in matrix form, such as images or structured signal arrays. In such cases, 2D CNNs can capture spatial relationships within the data, which might be indicative of complex respiratory patterns not easily discernible through 1D analysis. The architecture of a 2D CNN allows for the processing of these matrices, extracting features both horizontally and vertically across the input data.

Recurrent Neural Networks (RNNs), including their advanced variants like Long Short-Term Memory (LSTM) networks and Gated Recurrent Units (GRUs), excel in analyzing data where temporal dependencies are crucial. For respiratory event detection, where the sequence of events and their duration can provide critical diagnostic information, RNNs can be particularly effective. These models process signals in a sequence, capturing information from previous events to inform the analysis of subsequent data, thereby enhancing the detection of patterns over time.

Models incorporating self-attention mechanisms, such as the Transformer models, offer a significant advancement in handling sequences by enabling the model to weigh the importance of different parts of the input data dynamically. This mechanism allows the model to focus on more relevant segments of physiological signals, improving the accuracy and efficiency of detecting respiratory events. The self-attention mechanism is adept at handling long-range dependencies within the data, making it suitable for complex scenarios where interactions between distant events in the signal are significant.

In addition to neural networks, the system can also utilize decision-tree based models for the detection and quantification of respiratory events. Decision-tree models are a type of supervised learning algorithm that splits the data into subsets based on the value of input features, creating a tree-like model of decisions. The decision-tree model processes physiological signals by evaluating each feature's contribution to predicting respiratory events. For each segment of physiological data, the decision-tree model examines features such as SpO2, IHR, and PPG envelope signals. The tree structure allows the model to handle complex interactions between features, making decisions at each node to determine the likelihood of a respiratory event.

Each of these machine learning models can be integrated into the system either to replace or to complement the 1D CNN, depending on the nature of the physiological signals and the specific requirements of the respiratory event detection task. The ability to employ various types of neural networks enhances the system's adaptability, enabling customization for a broad range of applications. This flexibility enhances both the versatility and effectiveness of the system in various clinical and home monitoring environments.

The integration of multiple machine learning models into the system ensures that it can adapt to the evolving complexities of respiratory event detection, leveraging the strengths of each model type to achieve high accuracy and reliability in diagnosing sleep-disordered breathing conditions.

Below, we will provide a more in-depth introduction to the model threshold and vote threshold, both of which play pivotal roles in ensuring the accuracy and reliability of the event counting process. These thresholds are applied in a two-tiered approach to filter and validate the predictions made by the machine learning models, particularly ensuring that only genuine respiratory events are counted.

The model threshold is first applied during the initial analysis of each segment by the machine learning model 130. This model threshold is a predefined value that determines whether a given segment contains a respiratory event. The machine learning model 130, after analyzing a segment, generates a prediction score reflecting the likelihood of an event's presence. If this score surpasses the model threshold, the segment is marked as a positive detection, indicating a high probability of an event's occurrence within the segment. The determination of this model threshold is based on a careful analysis of historical data and extensive testing. It is set at a level that optimizes the balance between sensitivity (correctly identifying true events) and specificity (correctly dismissing non-events).

Following the application of the model threshold, the vote threshold comes into play as part of the voting mechanism. This mechanism is designed to aggregate the predictions across overlapping segments to corroborate and confirm the detection of respiratory events. Each segment that exceeds the model threshold contributes a vote towards the presence of an event in the overlapping time frame. The aggregated votes across multiple segments provide a robust measure of confidence in the event's occurrence.

The vote threshold is a critical value in this aggregation process. It is the minimum number of votes required for a respiratory event to be confirmed and counted. In essence, it is the level at which the aggregated predictions from multiple segments are deemed sufficient to verify an event's presence. This threshold is set to ensure that only events with strong and consistent indications across multiple model predictions are counted, thus minimizing the risk of false positives.

The voting mechanism operates by analyzing the overlapped segments. Each positive detection (where the prediction score exceeds the model threshold) contributes to the vote count within its overlapping time frame. When the total votes in any given time frame exceed the vote threshold, it is concluded that a respiratory event has occurred with high confidence. This event is then included in the final count.

In essence, the model threshold and vote threshold are significant for refining the event detection process. They work in tandem to filter out noise and errors, ensuring that the final event count is both accurate and reliable. The thresholds are set based on empirical data and expert judgment to capture the true occurrences of respiratory events, providing a solid foundation for the effective diagnosis and management of sleep-disordered breathing conditions.

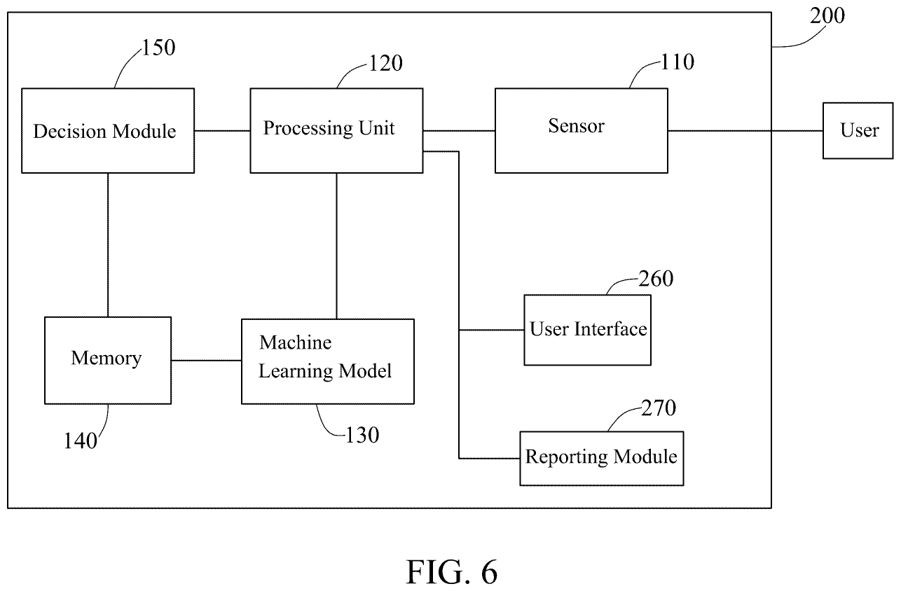

Please refer to FIG. 6, which illustrates another embodiment of the present invention. In contrast to the system 100 in FIG. 1, this embodiment's system 200 includes a user interface 260 and a reporting module 270. The user interface 260 and reporting module 270 of the system 200 are meticulously designed to ensure that the results of the respiratory event detection process are accessible, interpretable, and actionable for both clinicians and patients. Emphasizing user experience and clarity, these aspects of the system 100 play a significant role in translating complex physiological data and machine learning analyses into meaningful insights.

The user interface 260 of the system 200 is characterized by its simplicity and intuitiveness. For clinical use, the interface 260 is integrated into the existing medical monitoring platforms, offering seamless access to the respiratory event data alongside other vital patient information. It features a clean layout with easy-to-navigate controls, ensuring that medical professionals can quickly access and interpret the data without extensive technical training. Visual representations, such as graphs and timelines, display the occurrence and frequency of respiratory events throughout the monitoring period. These visual elements are accompanied by numerical data, providing a comprehensive view of the patient's respiratory activity.

For home users, the interface 260 is designed to be even more straightforward, often accessible through a web portal or a mobile application. It provides a summary of the night's sleep, highlighting any detected respiratory events and patterns. The user interface 260 also includes educational resources to help users understand the implications of their data and how it relates to their overall health and treatment progress.

The reporting model 270 of the system 200 is another critical aspect. For clinicians, the system 200 generates detailed reports that can be integrated into the patient's medical records. These reports include not only the count and timing of respiratory events but also trend analyses over time, which are essential for tracking the progression of the condition and the effectiveness of treatment. The reports can be customized to include additional metrics as required by the clinicians.

For home monitoring, the reporting model 270 offers simplified reports, focusing on key data points that are most relevant to the user. These reports can be configured to provide daily, weekly, or monthly summaries, giving users a clear view of their respiratory health trends over time. Users can also opt to share this data with their healthcare providers directly through the system, facilitating continuous monitoring and management of their condition.

The system 100, 200 for detecting and counting respiratory events, as described in this invention, can be applied in various real-world scenarios, each highlighting its utility and effectiveness. Let's explore some hypothetical examples and use cases that illustrate how this system operates in practical settings.

### Example 1: Clinical Sleep Study

In a clinical setting, a patient undergoing a sleep study for suspected sleep apnea is equipped with the sensor 110 of the system 100 and 200. Throughout the night, these sensors 110 continuously capture physiological signals such as SpO₂, IHR, and PPG envelope data. The system 100, 200 segments this data into overlapping segments with fixed length and analyzes each segment using its machine learning models. For instance, at one point in the night, the model identifies a significant drop in SpO₂ within the targeted window of a segment. The prediction score exceeds the model threshold, marking the segment as a positive detection of a respiratory event. Throughout the night, the decision module 150 aggregates these detections, confirming events when the aggregated scores surpass the vote threshold. In the morning, the system 100, 200 provides a comprehensive report detailing the number and timing of respiratory events, aiding the physician in diagnosing and treating the patient's condition.

### Example 2: Home Monitoring for Sleep Apnea Management

Consider an individual with diagnosed sleep apnea using the system 100, 200 at home to monitor the effectiveness of their treatment. The system's user-friendly design and non-invasive sensors 110 allow for easy setup and use during sleep. The machine learning model, trained to adapt to various respiratory patterns, analyzes the data for event detection. One night, the system 100, 200 detects several occurrences of abnormal breathing patterns, flagged as potential respiratory events. However, upon aggregation and application of the voting mechanism, only a few of these detections meet the vote threshold, indicating a lower frequency of significant events. This data provides valuable feedback to the individual and their healthcare provider, allowing them to assess the effectiveness of the current treatment regimen and make informed adjustments.

Additionally, while not the primary focus of this case, the preprocessing of input signals also holds a certain level of importance in the implementation of this system 100, 200 to ensure the signal quality of the input data. Recognizing that the reliability and accuracy of event detection are heavily contingent on the quality of the physiological signals captured, the system 100, 200 incorporates sophisticated methods to handle variations in signal quality.

Signal quality can be affected by a myriad of factors, including sensor placement, movement artifacts, and physiological variances among individuals. To address these challenges, the system 100, 200 first employs real-time signal quality assessment algorithms. These algorithms are adept at identifying segments of data that may be compromised due to poor sensor contact or external interference. By flagging these segments, the system 100, 200 ensures that only high-quality data is considered for further analysis.

Furthermore, the system 100, 200 utilizes advanced filtering techniques to mitigate the impact of transient noise and artifacts in the signal. These filters are designed to preserve the integrity of the physiological signals, retaining critical information while eliminating irrelevant or misleading data. The filtering process is carefully calibrated to strike a balance between removing noise and maintaining the fidelity of the signal.

In addition to filtering, the system 100, 200 employs adaptive algorithms that adjust to variations in signal characteristics. These algorithms are particularly important for accommodating physiological differences among patients and variations in sensor performance. By dynamically adjusting the parameters of the signal processing and analysis algorithms, the system ensures consistent performance across a wide range of signal conditions.

The system 100, 200 also incorporates redundancy in signal capture, using multiple sensors to obtain corroborative data. This redundancy provides a means to cross-validate the signals, enhancing the overall reliability of the data. If discrepancies arise between sensors, the system can identify and isolate unreliable data, relying on the more consistent readings for analysis.

To sum up, the signal quality management in the system 100, 200 is a multi-faceted approach involving real-time assessment, advanced filtering, adaptive algorithms, and sensor redundancy. Together, these methods ensure that the system consistently analyzes high-quality data, which is fundamental for the accurate detection and counting of respiratory events. This focus on signal quality not only bolsters the system's performance but also underscores its applicability in diverse settings, from controlled clinical environments to dynamic home monitoring situations.

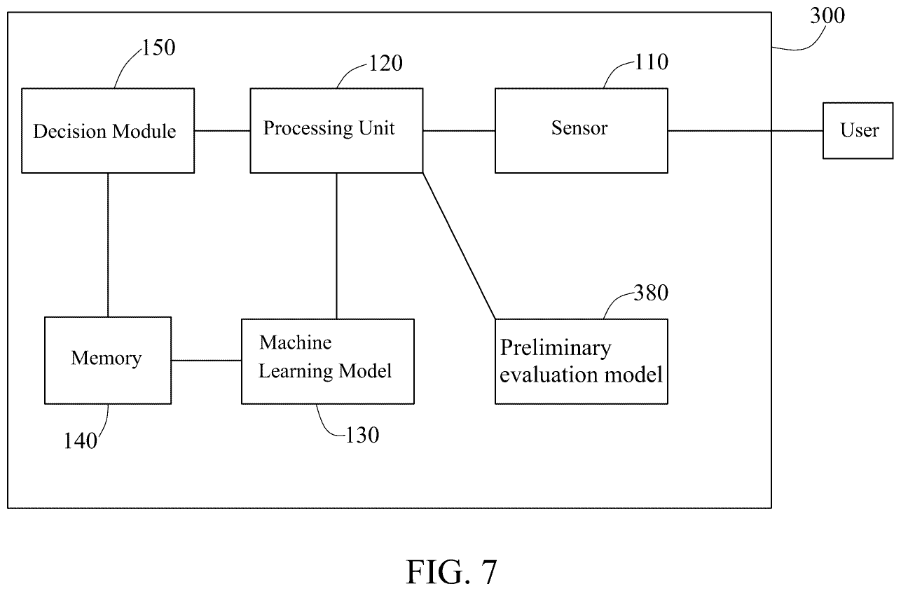

Please refer to FIG. 7, which illustrates third embodiment of the present invention. The system 300 further includes a preliminary evaluation model 380 designed to perform a preliminary evaluation of the patient's physiological signals recorded over the entirety of a night's sleep. In the embodiment, the preliminary evaluation model 380 is not utilized to parse detailed temporal sequences within the signal data; rather, its function is to categorize the patient's risk level for experiencing respiratory events.

The risk assessment conducted by the preliminary evaluation model 380 is based on a comprehensive analysis of the full night's signals. Upon generating a risk score, the patient is classified into one of several categories, such as ‘low’, ‘medium’, or ‘high’ risk, depending on the predetermined risk thresholds set within the model.

The classification of risk is critical as it directly informs the selection process for the model threshold and the final event count threshold utilized in the subsequent detailed event counting. The preliminary evaluation model 380's risk assessment allows for a tailored analytical approach in the subsequent analysis, where the sensitivity and specificity of event detection can be adjusted based on the initial risk classification. For instance, a higher risk classification may lead to the selection of more stringent thresholds to ensure that potential events are not overlooked, while a lower risk score may allow for less strict thresholds, reducing the likelihood of false positives.

This risk-based determination of thresholds aims to optimize the accuracy and efficiency of the system 300, especially considering the variability in signal characteristics and event prevalence among different patients. By integrating the risk classification into the event detection process, the systems ensures that the machine learning model is calibrated to the individual's specific signal profile, thereby enhancing the precision of respiratory event identification and quantification.

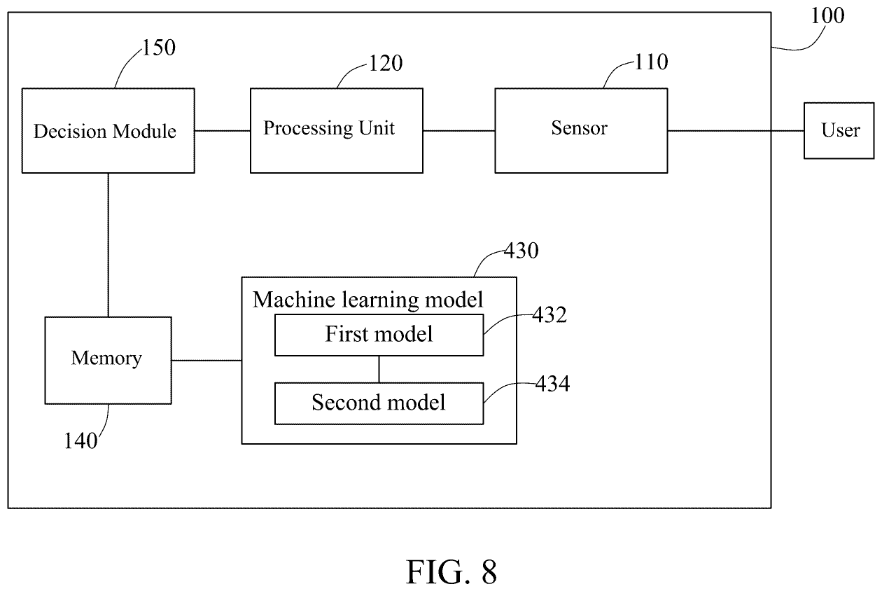

Please refer to FIG. 8, which illustrates fourth embodiment of the present invention. In the embodiment, the system 400 incorporates a sophisticated model ensembling technique as part of the analysis for the detection of respiratory events. This approach leverages the strengths of two distinct machine learning models to improve the overall accuracy of event prediction.

The machine learning model 430 includes the first model 432 in the ensemble operates on a larger scale, analyzing input segments of a first predetermined duration. This first predetermined duration, e.g. 300 seconds, is chosen based on the requirement to capture extended patterns within the physiological signal data that may indicate the presence of a respiratory event. By considering a broader view, the first model 432 is able to provide an initial probability score that a respiratory event has occurred within this larger time frame.

Conversely, the second model 434 is tasked with analyzing shorter input segments, e.g. 100 seconds. These segments are selected to allow for a more detailed examination of the physiological signals. The shorter duration enables the second model 434 to focus on more granular features that may be indicative of respiratory events, which might be missed when only considering larger segments.

The prediction scores generated by both models, i.e. the first model 432 and the second model 434, are then synthesized to form a final prediction score for respiratory event occurrence. This synthesis is performed through a weighted averaging process, wherein the prediction scores of the first model 432 and the second model 434 are combined according to predetermined weights. These weights are established based on the individual performance and reliability of each model, with the objective of emphasizing the contributions of the model that offers the most significant diagnostic value for the segment in question.

The final prediction score resulting from this weighted averaging is a refined assessment that benefits from the distinct analytical perspectives of both models. By utilizing the broad pattern recognition capability of the first model 432 and the detailed analysis of the second model 434, the system 400 is designed to accurately determine the presence of respiratory events.

This dual-model approach not only enhances the detection of respiratory events but also provides a robust framework capable of mitigating the individual weaknesses of each model. In practice, this means that the system 400 is less susceptible to the variances in signal quality or event characteristics that may affect a single-model approach. The ensemble method, therefore, represents a critical advancement in the field of physiological monitoring, offering improved reliability and diagnostic accuracy for patients undergoing evaluation for respiratory events.

This approach allows the system to dynamically adapt its parameters, ensuring that the machine learning model's performance is optimized for each patient on any given night. Consequently, the system can accommodate inter- and intra-patient variability, resulting in improved detection outcomes and more reliable monitoring of respiratory conditions.

The invention, as detailed in the preceding sections, stands as a robust and effective system for detecting and counting respiratory events. However, its design is not limited to the specific embodiments initially described. The system's architecture allows for various modifications and alternative embodiments, ensuring adaptability and applicability across different scenarios and technological advancements.

One significant area of variation lies in the machine learning model employed. While the primary embodiment utilizes one-dimensional convolutional neural network (1D CNNs) for their proficiency in handling time-series data, the system is not restricted to this model type. Alternative embodiments could incorporate different machine learning architectures such as recurrent neural networks (RNNs), especially Long Short-Term Memory (LSTM) networks, which are also well-suited for sequential data analysis. These models might offer advantages in certain contexts, particularly in handling longer sequence dependencies or in scenarios where the temporal dynamics of the respiratory signals are more complex.

Regarding sensor types, the primary embodiment focuses on PPG sensor to capture the physiological signals necessary for respiratory event detection. However, alternative embodiments of the system could integrate other sensor types. For instance, a person having ordinary skill in the art can combine the capnometry sensor and thermal sensor to ensure a comprehensive capture of respiratory metrics, enhancing the system's ability to accurately detect events. The capnometry sensor is instrumental in measuring the concentration of carbon dioxide (CO2) in exhaled air, providing real-time data on the respiratory cycle. The thermal sensor on the other hand, tracks the temperature changes associated with the inhalation and exhalation of air, offering another dimension of data to detect respiratory anomalies. In addition, the acoustic sensor could be employed to detect snoring or breathing sounds, while chest movement sensors could provide additional data on respiratory effort. The integration of these alternative sensors could enhance the system's capability to detect a broader range of respiratory events or improve its accuracy in specific environments.

The segmentation and voting process, significant to the system's operation, also presents avenues for variation. While the primary embodiment describes segments of a specific length with a defined overlap and a particular voting mechanism, these parameters can be modified to suit different requirements. For example, the segment length and overlap could be adjusted based on the nature of the physiological signals or the specific characteristics of the respiratory events being monitored. The voting mechanism itself could be adapted to incorporate different aggregation methods or thresholds, potentially improving system performance under varying conditions.

In conclusion, the invention is designed with flexibility in mind, allowing for variations and alternative embodiments that can adapt to different machine learning models, sensor types, and signal processing techniques. This adaptability not only broadens the scope of the invention but also ensures its relevance and applicability in the face of evolving technologies and diverse application requirements.

Although the invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

1. A method for detecting respiratory events in a subject, the method comprising:
receiving a time sequence of physiological signals from a sensor;
segmenting the time sequence into overlapping input segments, each segment comprising a predetermined duration;
analyzing each input segment with a machine learning model to generate a prediction score representing the probability of a respiratory event;
determining whether the prediction score for each input segment exceeds a model threshold;
aggregating the positive input segments across the time sequence; and
applying a final event count threshold to the aggregated positive input segments to produce a final event count.

2. The method of claim 1, wherein the step of applying a final event count threshold to the aggregated positive input segments to produce a final event count further comprises:
setting the final event count threshold as a predefined vote threshold;
applying a voting mechanism to the positive input segments identified based on the model threshold, wherein each positive input segment contributes a vote towards the presence of a respiratory event in the overlapping time frame; and
confirming the occurrence of a respiratory event when the number of votes for a specific time frame exceeds the vote threshold.

3. The method of claim 2, further comprising:
establishing the model threshold based on a training set of physiological signals wherein the presence and absence of respiratory events have been validated; and
adjusting the vote threshold based on a selected specificity and sensitivity for the detection of respiratory events.

4. The method of claim 2, wherein the vote threshold is applied to the aggregated positive input segments by averaging the prediction scores of overlapping segments and confirming the respiratory event when the average prediction score exceeds the vote threshold.

5. The method of claim 1, wherein the machine learning model is a one-dimensional convolutional neural network, a two-dimensional convolutional neural network, a recurrent neural network, a model with a self-attention mechanism, or a decision-tree based model.

6. The method of claim 1, wherein the predetermined duration of the input segments ranges from 10 seconds to 30 minutes.

7. The method of claim 1, wherein the prediction score is generated based on a localized portion of the input segment, said localized portion corresponding to a window within the predetermined duration is shorter than the input segments, which range from 1 seconds to 60 seconds.

8. The method of claim 1, wherein the physiological signals include at least one of: peripheral oxygen saturation, instantaneous heart rate, and photoplethysmogram envelope signals.

9. The method of claim 1, wherein the respiratory events are selected from the group consisting of apneas and hypopneas.

10. The method of claim 1, further comprising using a preliminary evaluation model to classify a patient's full night's signal into risk categories for determining the model threshold and the final event count threshold for respiratory event detection.

11. The method of claim 1, wherein the machine learning model comprises an ensemble including a first model with a first predetermined input segment duration and a second model with a second predetermined input segment duration which is shorter than the first predetermined input segment duration; wherein the prediction score for respiratory event occurrence is determined by a weighted average of the prediction scores from both the first model and the second model to enhance the accuracy of the respiratory event assessment.

12. A system for detecting respiratory events comprising:
a sensor configured to capture a time sequence of physiological signals from a subject;
a processing unit configured to segment the time sequence into overlapping input segments, each input segment comprising a predetermined duration;
a machine learning model implemented in the processing unit and configured to analyze each input segment to generate a prediction score representing the probability of a respiratory event;
a memory for storing a model threshold; and
a decision module, integrated within the processing unit, configured to:
determine whether the prediction score for each input segment exceeds the model threshold;
aggregate the positive input segments identified based on the model threshold; and
apply a final event count threshold to the aggregated positive input segments to confirm the occurrence of respiratory events and to produce a final event count.

13. The system of claim 12, wherein the memory additionally storing a vote threshold as the final event count threshold and the decision module is further configured to apply a voting mechanism to the aggregated positive input segments, wherein the confirmation of respiratory events and the final event count are based on whether the number of votes for specific time frames exceeds the vote threshold.

14. The system of claim 13, wherein the processing unit is further configured for:
establishing the model threshold based on a training set of physiological signals wherein the presence and absence of respiratory events have been validated; and
adjusting the vote threshold based on a selected specificity and sensitivity for the detection of respiratory events.

15. The system of claim 12, wherein the machine learning model is a one-dimensional convolutional neural network.

16. The system of claim 12, wherein the physiological signals include at least one of: SpO2, IHR, and PPG envelope signals, and wherein the system is configured to localize the prediction analysis to a specific window within each input segment.

17. The system of claim 12, further comprising a user interface configured to display the final event count along with the time sequence of physiological signals and indications of detected respiratory events.

18. The system of claim 12, wherein the sensor is a photoplethysmogram sensor, and wherein the photoplethysmogram sensor is configured to detect changes in blood volume related to the respiratory cycle, the machine learning model being further configured to analyze changes in photoplethysmogram signal characteristics.

19. The system of claim 12, further comprising a preliminary evaluation model configured to classify a patient's full night's signal into risk categories for determining the model threshold and the final event count threshold for respiratory event detection.

20. The system of claim 12, wherein the machine learning model comprises an ensemble including a first model with a first predetermined input segment duration and a second model with a second predetermined input segment duration which is shorter than the first predetermined input segment duration; wherein the prediction score for respiratory event occurrence is determined by a weighted average of the prediction scores from both the first model and the second model to enhance the accuracy of the respiratory event assessment.
